(19) United States
(12) Patent Application Publication
(10) Pub. No.: US 2010/0015074 A1
(43) Pub. Date: Jan. 21, 2010

(54) Cosmetic Makeup and/or Care Process

(75) Inventor: Xavier Blin, Paris (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/988,158

(22) PCT Filed: Jul. 13, 2006
(86) PCT No.: PCT/IB2006/052391

§ 371 Date: Mar. 10, 2008

Related U.S. Application Data
(60) Provisional application No. 60/712,105, filed on Aug. 30, 2005.

(30) Foreign Application Priority Data
Jul. 13, 2005 (FR) 05-52205

(51) Int. Cl. A61K 8/18; A61Q 1/02; A61Q 1/04

(52) U.S. Cl. 424/64; 424/63

(57) ABSTRACT

The present invention relates to a cosmetic process for making up and/or caring for keratin material(s), comprising the application, on contact with a keratin material, of a cosmetic composition comprising at least 75% by weight of compound(s) approved for food use relative to its total weight and having a covering power of greater than or equal to 30.

The present invention relates to a cosmetic makeup and/or care process comprising the application, to a keratin material, of a cosmetic makeup and/or care composition mainly composed of compounds approved for food use.

The cosmetic compositions toward which the present invention is directed are more particularly makeup and/or care products intended to be applied to the skin, the lips and/or the integuments, especially lipsticks, lip balms, lip pencils, liquid or solid foundations, especially cast as a stick or a dish, concealer products and skin coloring products, temporary tattoos, eye makeup products, for instance eye liners, in particular in the form of pencils, mascaras or eyeshadows. In general, these cosmetic compositions contain an appreciable amount of synthetic products, especially formulation additives to give them specific properties such as satisfactory stability over time and/or with regard to significant variations in temperature, satisfactory behavior especially in terms of makeup qualities, and good applicability.

However, in the cosmetics field where the turnover of products is extremely rapid, there is a constant need for novel formulation routes for obtaining a level of performance at least equal to that offered by the current products, or, in certain respects, even better.

What is more, the choice of raw materials used for the implementation of these novel formulation routes must be guided, as it has always been, by an ever-increasing concern to offer the maximum guarantee, both for the consumers for whom the cosmetic formulations that incorporate them are intended, and for preservation of the integrity of the environment.

Thus, according to one of its aspects, the present invention relates to a cosmetic process for making up and/or caring for keratin material(s), characterized in that it comprises the application to said keratin material of at least one cosmetic composition comprising at least 75% by weight, relative to its total weight, of compound(s) approved for food use, and which, according to one advantageous mode, may have a covering power of greater than or equal to 30.

According to another of its aspects, the present invention also relates to a cosmetic composition for making up and/or caring for keratin materials that is suitable for implementation of the process according to the invention.

According to one specific aspect of the invention, the compounds approved for food use may be “food grade compounds”.

In particular, the process of the invention may be a make up process.

In general, the compositions according to the invention contain a physiologically acceptable medium.

The term “cosmetic composition” denotes a composition as defined in Directive 93/35/EEC of the Council of 14 Jun. 1993.

The term “physiologically acceptable medium” denotes a nontoxic medium that may be applied to at least one human keratin material.

The term “keratin materials” covers the skin, mucous membranes, for instance the lips, the nails and keratin fibers, such as the eyelashes and the hair. The cosmetic compositions in accordance with the present invention are particularly advantageous for use on the skin and the lips.

More generally, according to the invention, the term “compounds approved for food use” means compounds chosen from ozokerite, rice wax, compounds referenced in the Codex alimentarius and materials consisting exclusively of compounds referenced in the Codex alimentarius, for example such as pearls.

The Codex Alimentarius, or Food Code, is the world reference acting as the authority for consumers, producers and processors of foodstuffs, national food control bodies and the international food products market.

It combines the food standards, guidelines and other codes of use established in the field of food products by the Commission of the Codex Alimentarius, created in 1963 by the Food and Agriculture Organization (FAO) and the World Health Organization (WHO), in the context of the FAO/WHO joint program on food standards and under the aegis of the WHO.

For the purposes of the invention, a compound referenced in the Codex alimentarius denotes a compound whose use as a food ingredient is considered in the Codex, and is or is not regulated therein according to specific terms.

It is understood that when terms are specified in the Codex alimentarius for certain ingredients, they are not decisive for the implementation of these same compounds in the compositions according to the invention.

In general, the term “food” ingredient denotes any substance other than water, used in the manufacture or preparation of a food and present in the finished product even though occasionally in a modified form. Thus, the term “food ingredient” especially includes food additives and food extracts.

Moreover, the term “food additive” means any substance that is not normally consumed as a foodstuff per se and is not normally used as a characteristic ingredient of a food, whether or not it has nutrient value, and whose deliberate addition to the foodstuff for a technological or organoleptic purpose, at any step in the manufacture, transformation, preparation, processing, packaging, wrapping, transportation or storage of this foodstuff, leads or may lead (directly or indirectly) to its incorporation or to the incorporation of derivatives thereof into the foodstuff or can affect in any other way the characteristics of this foodstuff.

For the purposes of the invention, a product consisting exclusively of compounds referenced in the Codex alimentarius denotes a material whose composition consists exclusively of compounds referenced in the Codex alimentarius and which, consequently, comprises at least two compounds, or even more, referenced in the Codex alimentarius. Materials of multilayer structure, for instance pearls, are especially covered under this definition. Thus, pearls generally consist of a mineral substrate such as mica or TiO₂ covered, for example, with a coat of iron oxide.

The Codex alimentarius under consideration according to the invention is that available at the date of filing of the present patent application.

For example, as regards the additives, it is the Codex Food Additive and Contaminants CX/FAC 05/37/6.

As regards extracts, these are especially defined in the Codex alimentarius volume 8 and more particularly in the Codex standards 19, 33 and 210.

For plant oils, the Codex under consideration is the version of the Codex standard 210 as amended in 2003.

As regards edible fats and oils, the Codex under consideration is the version of the Codex standard 19-1981, as revised in February 1993. For example, for olive oil, reference may be made more particularly to the version of Codex standard 33, revised in 1989.

According to one embodiment, the “compounds approved for food use” according to the present invention are “food grade compounds”.

According to this embodiment, a “food grade compound” may be a “compound approved for food use” that is sold as food grade compounds, i.e. a compound for which the supplier states it can be used in a food product. For example, a food grade compound is a compound specifically made to match the needs of the food industry. A food grade compound can consist only of edible products.

According to the invention, a “food grade compound” can also refers to a compound, specifications of which are given in the US Code of Federal Regulation Title 21 Chap I Parts 73, 74, 82, 172, 184 and 854.

In the US Code of Federal Regulation Title 21 Chap I, each chemical compound is defined as a “food grade compound” by a specification comprising i) a chemical name, ii) impurity levels the compound can contain, and/or iii) the concentration at which it can be used in a food.

In one embodiment of the invention, the food grade compound will be used in the cosmetic composition with the specifications given in US Code of Federal Regulation Title 21 Chap I.

As regards food grade oils, the part under consideration is 854.

As regards food grade semi-solid and solid fats like for example waxes, the part under consideration is 184.

As regards food grade additives, and more particularly food grade dyestuffs, the parts under consideration are 172, 73, 74 and 82.

More preferentially, the compositions in accordance with the invention comprise at least 80% by weight, especially at least 85% by weight, in particular at least 90% by weight, especially at least 95% by weight, and more particularly consist of about 100% by weight, relative to their total weight, of compound(s) approved for food use.

In one embodiment, the compositions in accordance with the invention comprise at least 50% by weight, in particular at least 60% by weight, especially at least 70% by weight, especially at least 80% by weight, especially at least 90% by weight, and more particularly consist of about 100% by weight, relative to their total weight, of food grade compound(s).

Unexpectedly, the presence of the compounds under consideration according to the invention does not prove to be harmful to the expected associated qualities for a cosmetic composition, for example as regards wear or gloss in the case of lipsticks, or covering power in the more particular case of foundations.

Covering Power

Advantageously, the cosmetic compositions according to the invention may have a covering power of greater than or equal to 30, in particular greater than or equal to about 50, especially greater than or equal to about 60, more particularly greater than or equal to about 80 and especially ranging from 90 to 100, or even of about 100.

This covering power may be measured via the following method.

In the case of a stick, the composition is blended beforehand so as to obtain a viscous paste.

In the case of a powder, 50 parts by weight of the powder are blended with 50 parts by weight of dimethicone (DC 200 Fluid 5CST from Dow Corning) so as to obtain a viscous paste.

The formulation is then spread to a thickness of 50 μm on an Erichsen type 24/5 contrast card, with a black background and a white background, and the trichromatic coordinates (X, Y and Z) are measured using a CH-2002 or CR-3700 colorimeter.

Similar spreadings are made on two other contrast cards and three measurements are taken on each card. The mean corresponding to these nine measurements is then calculated.

The covering power is equal to 100×Yb/Yw where Yb is the mean value of Y on the black background and Yw is the mean value of Y on the white background. A covering power of 100 corresponds to a totally opaque formulation.

Fatty Phase

The cosmetic compositions in accordance with the present invention may comprise a fatty phase especially comprising at least one compound chosen from oils and fatty substances that are solid at room temperature (20-25° C.) and atmospheric pressure, for example such as waxes and pasty fatty substances, and mixtures thereof.

Oils and solid fatty substances and mixtures thereof in a form suitable for human consumption, whether or not they have been subjected to conversions such as transesterification, hydrogenation or fractionation, are thus most particularly suitable for the invention.

Oils and solid fatty substances are especially foodstuffs in accordance with the definition in section 1 of the Codex alimentarius and composed of fatty acid glycerides.

According to one embodiment of the instant invention, the oils and solid fatty substances are food grade compounds.

They are more particularly listed in the US Code of Federal Regulation Title 21 Chap I Parts 172, 184 and 854.

They may be of animal, plant, mineral, synthetic or marine origin.

They may contain a small amount of other lipids, for instance phosphatides, unsaponifiable constituents and free fatty acids naturally present in these solid fatty substances and oils.

a) Oil

The term “oil” means any fatty substance that is in liquid form at room temperature (20-25° C.) and atmospheric pressure. The liquid fatty phase may also contain, besides oils, other compounds dissolved in the oils, such as gelling and/or structuring agents.

The cosmetic composition according to the present invention may comprise at least one and in particular at least two oils.

The oils that are suitable for the preparation of the cosmetic compositions according to the invention may be volatile or nonvolatile oils.

For the purposes of the present invention, the term “volatile oil” means an oil (or nonaqueous medium) capable of evaporating on contact with the skin in less than one hour, at room temperature and atmospheric pressure. The volatile oil is a volatile cosmetic oil, which is liquid at room temperature, especially having a nonzero vapor pressure, at room temperature and atmospheric pressure, in particular having a vapor pressure ranging from 0.13 Pa to 40 000 Pa (10⁻³ to 300 mmHg), preferably ranging from 1.3 Pa to 13 000 Pa (0.01 to 100 mmHg) and preferably ranging from 1.3 Pa to 1300 Pa (0.01 to 10 mmHg).

For the purposes of the present invention, the term “nonvolatile oil” means an oil having a vapor pressure of less than 0.13 Pa.

The volatile or nonvolatile oils may be hydrocarbon-based oils especially of animal, mineral or plant origin, synthetic oils, silicone oils or fluoro oils, or mixtures thereof.

For the purposes of the present invention, the term “hydrocarbon-based oil” means an oil mainly containing hydrogen and carbon atoms and possibly oxygen, nitrogen, sulfur and/or phosphorus atoms.

The oils that are more particularly considered according to the invention are hydrocarbon-based oils and more preferentially edible oils especially referenced in the Codex alimentarius and more specifically in standards 19 to 27, 33, 34, 123 to 128 and 210 thereof.

As examples of oils that are suitable for use in the present invention, mention may be made of oils chosen from oils comprising at least one fatty acid chosen from caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid and nervonic acid, and a mixture thereof.

They are more particularly hydrocarbon-based plant oils and in particular those chosen from triglycerides consisting of fatty acid esters of glycerol, the fatty acids of which may have chain lengths ranging from C₄ to C₂₄, these chains possibly being linear or branched, and saturated or unsaturated. These oils are especially heptanoic or octanoic triglycerides, groundnut oil, babassu oil, coconut oil, grapeseed oil, cottonseed oil, corn oil, corn germ oil, mustard seed oil, palm oil, rapeseed oil, sesame seed oil, soybean oil, sunflower oil, wheatgerm oil, canola oil, apricot oil, mango oil, castor oil, shea oil, avocado oil, olive oil, sweet almond oil, peach kernel oil, walnut oil, hazelnut oil, macadamia oil, jojoba oil, alfalfa oil, poppy seed oil, pumpkin oil, marrow oil, blackcurrant oil, evening primrose oil, millet oil, barley oil, quinoa oil, rye oil, safflower oil, candlenut oil, passionflower oil, musk rose oil or shea butter oil, or alternatively caprylic/capric acid triglycerides, and mixtures thereof.

According to one embodiment, the compositions according to the instant invention will contain at least some canola oil, in particular when they are intended to confer a gloss effect.

For comfort reasons, the compositions according to the invention will contain reduced amounts of castor oil. In effect, the castor oil is inclined to be partially oxidized by air with time and thus may generate unpleasant smell. Accordingly, the compositions may preferably contain less than 5% by weight of castor oil, especially less than 2%, in particular less than 1% by weight relative to the total weight of the composition and more particularly are free of castor oil.

According to one particular embodiment, the cosmetic compositions in accordance with the invention may comprise at least one oil chosen from hydrocarbon-based plant oils and more particularly chosen from the following oils approved for food use: isopropyl myristate sold by Stearinerie Dubois, the caprylic/capric acid triglycerides sold by Stepan; hybrid rapeseed oil, liquid cottonseed oil, refined protected deodorized mango oil, the liquid fraction of protected shea butter and the refined canola seed oil sold by Karlshamns; Lipex Sheasoft and cottonseed oil sold by Karslshamns; the deodorized apricot kernel oil sold by Nestlé; the sweet almond oil sold by Soetenaey; the peach kernel oil sold by Aarhus United; rapeseed oil, corn germ oil, olive oil, grapeseed oil, soybean oil and sunflower oil sold by Huileries de Lapalisse and walnut oil sold by Soetenaey.

More preferably, these oil compounds are food grade compounds, as defined here-above.

According to one particular embodiment, the cosmetic composition in accordance with the present invention comprises at least one oil chosen from canola oil, caprylic/capric acid triglycerides, apricot oil, peach oil, walnut oil and olive oil.

According to one particular embodiment, the cosmetic compositions according to the invention comprise from 0.1% to 99% by weight, especially from 1% to 90% by weight, in particular from 5% to 70% by weight, in particular from 10% to 65% by weight and more particularly from 20% to 60% by weight, relative to the total weight of oil(s) approved for food use in the composition and more particularly referenced in the Codex alimentarius.

Besides the abovementioned oils, the compositions in accordance with the present invention may obviously comprise at least one other liquid fatty substance, with the proviso that it is present in amounts in accordance with the requirements according to the invention.

Nonvolatile hydrocarbon-based oils that may especially be mentioned include:
synthetic ethers containing from 10 to 40 carbon atoms;
linear or branched hydrocarbons of mineral or synthetic origin such as petroleum jelly, polydecenes, hydrogenated polyisobutene such as Parleam, and squalane, and mixtures thereof, and in particular hydrogenated polyisobutene,
synthetic esters, for instance oils of formula R₁COOR₂ in which R₁ represents a linear or branched fatty acid residue containing from 1 to 40 carbon atoms and R₂ represents a hydrocarbon-based chain that is especially branched, containing from 1 to 40 carbon atoms provided that R₁+R₂≧10.

The esters may be chosen especially from fatty acid esters, for example:
cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkylbenzoate, polyethylene glycol diheptanoate, propylene glycol 2-diethyldihexanoate and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, and hydroxylated esters, for instance isostearyl lactate and diisostearyl malate;
polyol esters and pentaerythritol esters, for instance dipentaerythrityl tetrahydroxystearate/tetraisostearate;
esters of diol dimers and diacid dimers such as Lusplan DD-DA5® and Lusplan DD-DA7®, sold by the company Nippon Fine Chemical and described in patent application FR 0302809 filed on Mar. 6, 2003;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol; and
dialkyl carbonates, the two alkyl chains possibly being identical or different, such as dicaprylyl carbonate sold under the name Cetiol CC® by Cognis.

The volatile hydrocarbon-based oils may be chosen from hydrocarbon-based oils containing from 8 to 16 carbon atoms, and especially branched C₈-C₁₆ alkanes (also known as isoparaffins), for instance isododecane (also known as 2,2,4,4,6-pentamethylheptane), isodecane, isohexadecane and, for example, the oils sold under the trade names Isopars® or Permethyls®.

The compositions according to the invention may also contain volatile or nonvolatile silicone oils.

The nonvolatile silicone oils that may be used in the composition according to the invention may be nonvolatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and dimethicones or phenyltrimethicones with a viscosity of less than or equal to 100 cSt, and mixtures thereof.

Volatile silicone oils that may more particularly be used include volatile linear or cyclic silicone oils, especially those with a viscosity≦8 centistokes (8×10⁻⁶ m²/s) and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made especially of dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoro-methylcyclopentane, and mixtures thereof, may also be used.

The compositions according to the invention may also advantageously comprise at least one compound chosen from waxes and pasty fatty substances, and mixtures thereof.

b) Waxes

The wax is solid at room temperature (25° C.), has a reversible solid/liquid change of state, has a melting point of greater than 30° C., which may be up to 200° C. and a hardness of greater than 0.5 MPa, and has an anisotropic crystal organization in the solid state.

It may be a hydrocarbon-based wax, fluoro wax and/or silicone wax and may be of animal, plant, mineral or synthetic origin.

Advantageously, the compositions in accordance with the present invention comprise at least one wax chosen from waxes approved for food use.

For the purposes of the present invention, a “wax approved for food use” covers ozokerite, rice wax, and the waxes referenced in the Codex alimentarius, more particularly including the waxes referenced in table 1 of the Codex alimentarius.

Thus, the compositions in accordance with the present invention advantageously comprise a wax chosen from beeswax, ozokerite, rice wax, carnauba wax, candelilla wax and microcrystalline waxes, and mixtures thereof.

According to one embodiment of the instant invention, the wax compounds are food grade compounds, as defined previously in the application, and more particularly are referenced in the US code of Federal Regulation Title 21 Chap I Part 184.

Advantageously, the wax used in the cosmetic compositions in accordance with the invention is chosen from the microcrystalline wax sold by Paramelt and more particularly ozokerite, beeswax, candelilla wax or carnauba wax sold by Strahl & Pitsch, and mixtures thereof.

These waxes are preferably used under a form specified by the supplier as being convenient for food industry.

According to one particular embodiment, the wax(es) approved for food use is or are present in the cosmetic compositions in accordance with the present invention in a content ranging from about 1% to about 50%, in particular from about 3% to about 40%, in particular from about 5% to about 30% and especially from about 7% to about 20% by weight relative to the total weight of the compositions.

Besides these waxes approved for food use, the compositions according to the invention may comprise one or more waxes chosen, for example, from synthetic waxes, for instance polyethylene wax (preferably with a molecular weight of between 400 and 600) or Fischer-Tropsch waxes, silicone waxes, for instance alkyl or alkoxy dimethicones containing from 16 to 45 carbon atoms, paraffin waxes, ceresins, for instance isoparaffins with a melting point of less than 40° C., such as EMW-0003, sold by the company Nippon Seirou, α-olefin oligomers, such as the polymers Performa V® 825, 103 and 260, sold by the company New Phase Technologies; ethylene-propylene copolymers, such as Performalene® EP 700, and mixtures thereof.

c) Pasty Compounds

The cosmetic compositions in accordance with the present invention may also comprise at least one pasty compound.

For the purposes of the present invention, the term “pasty” refers to a fatty compound with a reversible solid/liquid change of state and comprising, at a temperature of 25° C., a liquid fraction and a solid fraction. The term pasty also means polyvinyl laurate.

Polyol esters are most particularly suitable as pasty compounds according to the invention.

The polyol esters that may be used in the context of the present invention are commercially available or may be prepared in a conventional manner. They are generally of plant origin and may be obtained especially by mono- or polyesterification of a polyol with a C₂-C₃₄ monocarboxylic acid, for instance a fatty acid or with a dicarboxylic acid such as a diacid dimer.

The ester obtained may especially be a polyester, a triester, a diester, a monoester or a mixture thereof. In the present case, the ester may be a mixture of two or more types of ester formed with different carboxylic acids.

In the case of esterification with a monocarboxylic acid, esters with a relatively high molecular weight, ranging from about 200 to 1300 g/mol, may be obtained.

In the esterification reaction with a dicarboxylic acid, a polyol dicarboxylate may be obtained, which has a weight-average molecular weight, determined by gel permeation chromatography (GPC), ranging from 200 to 20 000 g/mol and preferably between 2000 and 4000 g/mol.

For the purposes of the present invention, the terms “polyol” and “polyhydric alcohol” should be understood as meaning any organic molecule comprising at least two free hydroxyl groups.

The polyhydric alcohols that are advantageously suitable for the formulation of the cosmetic compositions according to the present invention are those especially containing from 2 to 20 carbon atoms, in particular from 3 to 10 carbon atoms and more particularly from 4 to 6 carbon atoms.

Advantageously, the polyol may be chosen, for example, from a diol dimer, glycerol, propylene glycol, butylene glycol, pentylene glycol, hexylene glycol, dipropylene glycol, diethylene glycol, sorbitol, hydroxypropyl sorbitol and 1,2,6-hexanetriol; glycol ethers (especially containing from 3 to 16 carbon atoms) such as mono-, di- or tripropylene glycol (C₁-C₄)alkyl ethers and mono-, di- or triethylene glycol (C₁-C₄)alkyl ethers; and mixtures thereof.

It may also be a “diol dimer”, i.e. saturated diols produced by hydrogenation of the corresponding diacid dimers.

A diol dimer may be produced by hydrogenation of a diacid dimer, which is itself obtained by dimerization of an unsaturated fatty acid especially of C₈ to C₃₄, such as those mentioned previously, especially of C₁₂ to C₂₂ and in particular of C₁₆ to C₂₀, preferably C₁₈ such as, for example, oleic acid and linoleic acid.

The polyols that are more particularly suitable are sugars chosen from monosaccharides, disaccharides and trisaccharides. Illustrations of these sugars that may especially be mentioned include monosaccharides such as xylose, arabinose, galactose, fructose, mannose and glucose, and mixtures thereof. Illustrations of disaccharide polyols that may more particularly be mentioned include maltose, lactose and sucrose and combinations thereof.

The monocarboxylic acid that may be used in the present invention may contain from 2 to 34 carbon atoms and especially from 10 to 32 carbon atoms.

By way of illustration of monocarboxylic acids that are suitable for the invention, mention may be made especially of:
saturated linear acids such as butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, docosanoic acid and tetracosanoic acid,
branched fatty acids, for instance isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyldodecanoic acid, 2-decyltetradecanoic acid, 2-dodecylhexadecanoic acid, 2-tetradecyloctadecanoic acid, 2-hexadecyloctadecanoic acid and long-chain fatty acids obtained from lanolin,
unsaturated linear C₈ to C₃₄ fatty acids, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid and arachidonic acid,
hydroxy acids such as 2-hydroxybutanoic acid, 2-hydropentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxytetradecanoic acid, 2-hydroxyhexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid and 2-hydroxytetracosanoic acid,
cyclic acids such as cyclohexanoic acid, hydrogenated rosin, rosin, abietic acid, hydrogenated abietic acid, benzoic acid, p-oxybenzoic acid, p-aminobenzoic acid, cinnamic acid, p-methoxycinnamic acid, salicylic acid, gallic acid, pyrrolidonecarboxylic acid and nicotinic acid, and
fatty acids of natural origin, such as the fatty acids of orange oil, of avocado oil, of macadamia oil, of olive oil, of hydrogenated soybean oil, of jojoba oil, of palm oil, of castor oil, of wheatgerm oil, of saffron oil, of cottonseed oil and of mink oil, and mixtures thereof.

It is more particularly a fatty acid, especially as defined above.

The dicarboxylic acid that may be used according to the invention should contain at least two carboxylic groups per molecule.

It may be represented especially by the formula below:

HOOC—(CH₂)ₙ—COOH

in which n is an integer from 1 to 16 and preferably from 3 to 16.

As non-limiting illustrations of dicarboxylic acids that are suitable for the invention, mention may be made especially of malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradecamethylenedicarboxylic acid, 1,15-pentadecamethylenedicarboxylic acid and 1,16-hexadecamethylenedicarboxylic acid, and mixtures thereof.

The dicarboxylic acid may also be a diacid dimer. The term “diacid dimer” denotes a diacid obtained by polymerization reaction, especially by intermolecular dimerization of at least one unsaturated fatty acid especially of C₈ to C₃₄, such as those mentioned previously, especially of C₁₂ to C₂₂ and in particular of C₁₆ to C₂₀, preferably of C₁₈ such as, for example, oleic acid and linoleic acid.

Polyol polyesters in which the fatty acid ester units of the polyester comprise saturated or unsaturated chain lengths chosen such that the compound has the required behavior in terms of pasty compounds according to the invention, are also most particularly suitable as polyol esters.

The unsaturated fatty acid chains are typically branched chains and more particularly contain from 12 to about 22 and more particularly from about 18 to 22 carbon atoms.

The unsaturated fatty acid chains more particularly considered are monounsaturated and/or diunsaturated C₁₈ fatty acids.

These long chains may be combined with shorter saturated fatty acid chains. They are generally linear and contain from 2 to about 12, preferably from 6 to about 12 and more particularly from 8 to 12 carbon atoms.

In general, the degree of esterification of these fatty acid esters is such that about 60% of the hydroxyl functions of the polyols and more particularly about 85% or even 95% of the hydroxyl functions of the polyols are esterified.

As regards the unsaturated long-chain fatty acid ester units, mention may be made more particularly of lauroleates, myristoleates, palmitoleates, oleates, elaidates, eructates, linoleates, linolenates, arachidonates, eicosapentaenoates and docosahexaenoates. For reasons of stability to oxidation, monounsaturated and diunsaturated fatty acid chains are preferred.

As regards the long-chain unsaturated fatty acid ester units, mention may be made more particularly of arachidate, behenate, linoserate and serotate esters.

As regards the short-chain saturated fatty acid ester units, they may be more particularly acetate, caproate, caprylate, caprate and laurate.

As solid polyol fatty acid polyesters that are most particularly suitable for the invention, mention may be made more particularly of raffinose octaesters in which the esterifying fatty acid parts are linoleate and behenate, maltose hectaesters in which the esterifying fatty acid parts are derived from sunflower seed oil fatty acid and from lignoserate, sucrose octaesters in which the esterifying fatty acid parts are behenate and oleate, and sucrose octaesters in which the esterifying fatty acid parts are laurates, linoleates and behenates.

Such solid fatty acid polyesters may be obtained according to methods already described for the preparation of the polyol polyesters. In this respect, reference may be made especially to documents U.S. Pat. No. 5,306,516, U.S. Pat. No. 5,306,515, U.S. Pat. No. 5,305,514, U.S. Pat. No. 4,797,300, U.S. Pat. No. 3,963,699, U.S. Pat. No. 4,518,772 and U.S. Pat. No. 4,517,360.

According to one embodiment, the composition according to the invention comprises at least one ester of at least one carboxylic acid containing 1 to 7 carbon atoms and of a polyol containing at least 4 hydroxyl groups, the said ester having for example a molecular mass of less than 5 000 g/mol.

The ester preferably has a molecular mass of less than 2 000, more preferably less than 1 000, more preferably still less than 900 g/mol. The molecular mass of the ester is preferably greater than 100 g/mol.

Such esters are particularly interesting for enhancing the wear of the compositions incorporating them.

The polyol according to the invention may be a monosaccharide, a polyhydroxyaldehyde (aldose) or polyhydroxyketone (ketose), which is cyclized or not. The polyol is preferably a monosaccharide cyclized in hemiacetal form.

The polyol may also be a polyol derived from a monosaccharide, such as eythritol, xylitol or sorbitol.

Among aldoses mention may be made of D-ribose, D-xylose, L-arabinose, D-glucose (or alpha-D-glucopyranose when in cyclic hemiacetal form), D-mannose and D-galactose.

Among ketoses mention may be made of D-xylulose and D-fructose (or beta-D-fructofuranose when in cyclic hemiacetal form).

The polyol may be a monosaccharaide or a polysaccharide containing from 1 to 10 monosaccharide units, preferably from 1 to 4, more preferably 1 ot 2 monosaccharide units.

The polyol is preferably selected from erythritol, xylitol, sorbitol, glucose and sucrose.

The polyol according to the invention is preferably a disaccharide. Among disaccharides mention may be made of sucrose (alpha-D-glucopyranosyl-(1-3)-beta-D-fructofuranose), lactose (beta-D-galactopyranosyl-1(1-4)-beta-D-glucopyranose) and maltose (alpha-D-glucopyranosyl-(1-4)-beta-D-glucopyranose).

The polyol may be a polysaccharide composed of two or more identical monosaccharide units or at least two different monosaccharide units. The ester according to the invention may be composed of a polyol substituted by at least two different monocarboxylic acids or by at least three different monocarboxylic acids.

The ester may be obtained by copolymerizing two esters according to the invention, in particular by copolymerizing i) a sucrose substituted by benzoyl groups and ii) a sucrose substituted by acetyl and/or isobutyryl groups.

The ester preferably contains no polar group, in particular no hydroxyl group. In other words, during the esterification reaction between the acid and the polyol, the acid is added in an amount sufficient to react with all of the hydroxyl groups of the polyol. The polar groups are, for example, ionic or non-ionic polar groups selected from —COOH; —OH; ethylene oxide; propylene oxide; —PO₄; —NHR; —NR₁R₂ with R₁ and R₂ optionally forming a ring and each representing a linear or branched C₁ to C₂₀ alkyl or alkoxy radical.

The acid is preferably a monocarboxylic acid containing 1 to 7 carbon atoms, preferably 1 to 5 carbon atoms. It may be selected in particular from acetic, n-propanoic, isopropanoic, n-butanoic, isobutanoic, tert-butanoic, n-pentanoic and benzoic acids.

The ester may be obtained from at least two different monocarboxylic acids.

In one embodiment the acids is an unsubstituted linear or branched acid.

The acid is preferably selected from acetic acid, isobutyric acid and benzoic acid.

In one preferred embodiment the ester has sucrose diacetate hexa(2-methylpropanoate) as a chemical name, and can bear the INCI name sucrose acetate isobutyrate.

As pasty compounds that are advantageously suitable for the formulation of the cosmetic compositions in accordance with the present invention, mention may be made more particularly of fractionated hydrogenated triglycerides and especially those sold by SIO; hydrogenated plant oils, hydrogenated palm oil, cocoa butter and, for example, those sold by Karlshamns, solid cottonseed oil, for example the oil sold by SIO, and sucrose acetate isobutyrate, for example the product sold by Eastman Chemical.

Among the other pasty compounds that may be used in the composition according to the invention, mention may also be made of lanolines and lanoline derivatives, for instance acetylated lanolines, oxypropylenated lanolines or isopropyl lanolate, and mixtures thereof. However, the amount in lanoline(s) will be also adjusted for avoiding any undesirable effect in particular in term of smell as soon as these compounds are inclined to oxidize with time.

Mention may also be made of pasty silicone compounds such as high molecular weight polydimethylsiloxanes (PDMS) and in particular those with pendent chains of the alkyl or alkoxy type containing from 8 to 24 carbon atoms, and a melting point of 20-55° C., for instance stearyl dimethicones, especially those sold by the company Dow Coming under the trade names DC2503® and DC25514®, and mixtures thereof.

According to one variant of the invention, the solid substances, of wax type, may be chosen for their efficacy in texturing a liquid fatty phase. In general, the compounds that are suitable in this respect have a melting point of greater than or equal to 50° C., in particular greater than or equal to 55° C. or even ranging from 55 to 150° C. and even from 60 to 130° C.

In accordance with a preferred embodiment, the compositions according to the invention contain
at least one hydrocarbon-based plant oil like for example canola oil, caprylic/capric acid triglycerides and/or one of their mixtures and
at least one ester of at least one carboxylic acid containing 1 to 7 carbon atoms and of a polyol containing at least 4 hydroxyl groups, in particular the sucrose diacetate hexa(2-methylpropanoate) and
at least one hydrogenated vegetable oil.

According to a more specific embodiment, the compositions according to the invention contain
about 10 to 40% by weight of hydrocarbon-based plant oil(s) in particular non hydrogenated vegetable oil(s),
about 20 to 70% by weight of ester(s) of at least one carboxylic acid containing 1 to 7 carbon atoms and of a polyol containing at least 4 hydroxyl groups, and
about to 10 to 40% by weight of hydrogenated vegetable oil(s)
   The percentages being expressed relative to the total weight of the mixture of said compounds.

Naturally, such compositions may further contain at least one compound selected among waxes, dyestuffs and fillers as described here above, like for example rice starch and mixtures thereof.

Aqueous Phase

The composition according to the invention may also comprise at least one aqueous medium, constituting an aqueous phase, which can form the continuous phase of the composition.

The aqueous phase may consist exclusively of water.

It may also comprise a mixture of water and of water-miscible organic solvent (miscibility in water of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes.

According to one embodiment, the cosmetic composition that is suitable for use in the present invention may be in the form of a simple emulsion, a water-in-oil or oil-in-water emulsion, a multiple emulsion (water-oil-water or oil-water-oil) or an inverse emulsion, the use of which is well known to those skilled in the art.

The aqueous phase (water and optionally the water-miscible organic solvent) may be present in a content ranging from 0.1% to 25% by weight, especially ranging from 0.1% to 20% by weight and in particular from 0.1% to 10% by weight relative to the total weight of the composition.

According to yet another aspect of the invention, the composition according to the invention may be anhydrous.

For the purposes of the present invention, the term “anhydrous composition” means a composition comprising less than 10% by weight of water relative to the total weight of the composition, especially less than 5%, in particular less than 2% and more particularly less than 1% by weight of water relative to the total weight of the composition.

Advantageously, an anhydrous composition according to the invention is free of water.

Dyestuffs

The cosmetic composition in accordance with the invention may advantageously incorporate one or more dyestuffs, especially such as pigments or pearls conventionally used in cosmetic compositions.

The term “pigments” should be understood as meaning white or colored, mineral or organic particles, which are insoluble in an aqueous solution and which are intended to color and/or opacify the corresponding cosmetic composition.

As mineral pigments that may be used in the invention, mention may be made of zirconium oxide or cerium oxide and also zinc oxide or chromium oxide, ferric blue, manganese violet, ultramarine blue and chromium hydrate.

The term “pearls” should be understood as meaning iridescent or noniridescent colored particles of any form, produced especially by certain mollusks in their shell or else synthesized, and which have a color effect by optical interference.

These dyestuffs may be present in a proportion of from 0.01% to 40% by weight, especially from 0.1% to 20% by weight and in particular from 0.5% to 15% by weight, or even from 1% to 10% by weight, relative to the total weight of the cosmetic composition.

Advantageously, the compositions according to the invention comprise dyestuffs approved for food use especially referenced in the Codex alimentarius and more specifically the substances referenced in table 1 thereof.

In particular, the dyestuffs may be dyestuffs containing at least two materials, said dyestuff being referenced in the Codex alimentarius or consisting exclusively of materials referenced in the Codex alimentarius.

More specifically, it is at least one dyestuff chosen from pearls consisting of materials referenced in the Codex alimentarius, lakes approved for food use, and also referenced in the Codex alimentarius, and coloring substances approved by the Codex alimentarius, and mixtures thereof.

Illustrations of these dyestuffs that may be mentioned more particularly include mineral pigments such as titanium oxides and iron oxides, water-soluble or liposoluble coloring agents, for instance Sudan red, β-carotene, beetroot juice, the disodium salt of ponceau, the disodium salt of alizarine green, quinoline yellow, DC Red No. 7, DC Green No. 6, DC Yellow No. 11, DC Violet No. 2, DC Orange No. 5, the trisodium salt of amaranth, the disodium salt of tartrazine, the monosodium salt of rhodamine, the disodium salt of fuchsin, xanthophyll, canthaxanthine, carmines, erythrosin, indigotin and riboflavin.

In the case of the present invention, the choice of pearls consisting of materials referenced in the Codex alimentarius is preferred.

Illustrations of such pearls that may be mentioned more particularly include pearls based on mica coated with titanium and/or with iron oxide, pearls based on mica coated with titanium and/or with iron oxide and surface-coated with at least one organic dye, for instance carbon black, and pearls based on mica coated with aluminum, silver and/or gold and, where appropriate, surface-coated with at least one organic dye.

Advantageously, the base material and the abovementioned surface coatings are materials approved for food use. For example TiO₂ is authorized under the reference E171, iron oxide under the reference E172, carbon black under the reference E153, aluminum under the reference E174, and gold under the reference E175.

Such composite materials are especially sold by the company Merck under the name Candurin®.

As regards lakes, mention may be made more particularly of carbon black, pigments of the type such as organic barium, strontium, calcium, aluminum or titanium lakes, including those submitted for certification by the Food and Drug Administration (FDA) (for example FD & C), lakes based on cochineal carmine, or the diketopyrrolopyrroles (DPP) described in documents EP-A-542 669, EP-A-787 730, EP-A-787 731 and WO-A-96/08537.

As pigments of “lake” type that are most particularly suitable for the invention, mention may be made especially of those sold by LCW Sensient under the names FD&C Yellow No. 5/E102, FD&C Yellow No. 6/E110, FD&C Blue No. 1/E132, FD&C Red No. 40/E129, FD&C Blue No. 2 aluminum lake, FD&C Yellow No. 5 aluminum lake, FD&C Yellow No. 6 aluminum lake, FD&C Blue No. 1 aluminum lake, FD&C Red No. 40 aluminum lake and FD&C Green No. 3 aluminum lake.

FD&C Blue No. 2 aluminum lake, FD&C Yellow No. 5 aluminum lake, FD&C Yellow No. 6 aluminum lake, FD&C Blue No. 1 aluminum lake, FD&C Red No. 40 aluminum lake and FD&C Green No. 3 aluminum lake are most particularly suitable for the invention.

As regards the lakes and pearls, these dyestuffs are especially advantageous for affording an effect other than a simple conventional shade effect, i.e. a unified and stabilized effect as produced by standard dyestuffs, for instance monochromatic pigments.

For the purposes of the invention, the term “stabilized” means free of a color variability effect according to the angle of observation. The effect obtained with the pearls and/or lakes may be an effect chosen from metallic effects, and especially a mirror, soft-focus and/or rainbow effect.

Besides the abovementioned coloring agents such as those specifically approved for food use, the compositions may obviously comprise other organic or inorganic coloring substances.

They may thus be organic pigments. Mention may especially be made of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11.

The organic dyestuff may comprise an organic lake supported on an organic support such as colophony or aluminum benzoate, for example. Among the organic lakes that may be mentioned in particular are those known under the following names: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake.

The compositions according to the invention may also contain diffracting agents, goniochromatic agents and/or reflective particles.

According to a one embodiment, the compositions according to the instant invention contain dyestuff considered as being food grade compound as defined here-above.

More particularly dyestuff can be chosen from dyestuff having the specifications given in the US Code of Federal Regulation Title 21 Chap I parts 73, 74 and 82.

According to a one embodiment, the dyestuff will in particular be made of at least 50% by weight, more particularly at least 75% by weight, especially 90% by weight of the total weight, of “food grade compounds” as defined here-above.

According to a specific embodiment, these compositions may be free of non food grade dyestuff.

Filler

The cosmetic compositions also generally contain fillers of mineral or organic origin.

Needless to say, compounds proposed above, especially as coloring agent, are capable of jointly fulfilling this function.

Nonpigmentary inorganic compounds approved by the Codex alimentarius and more particularly referenced in table 1 thereof are most particularly suitable for the invention.

In this respect, mention may be made more particularly of talc, precipitated calcium carbonate, magnesium carbonate, rice starch, and magnesium hydrogen carbonate.

Additives

The compositions according to the invention may also comprise any ingredient conventionally used as additives in cosmetics and dermatology.

These additives are advantageously chosen from the food additives proposed in table 1 of the Codex alimentarius, for example antioxidants, thickeners, sequestrants, acidifying or basifying agents and preserving agents, and mixtures thereof.

The compositions according to the invention may also contain flavorings and/or fragrances.

The amounts of these various ingredients are those conventionally used in the fields under consideration and range, for example, from 0.01% to 20% by weight relative to the total weight of the composition.

Needless to say, a person skilled in the art will take care to select this or these optional additional compound(s), and/or the amount thereof, such that the advantageous properties of the product according to the invention are not, or are not substantially, adversely affected by the addition under consideration.

Gloss Test

Advantageously, the cosmetic compositions according to the invention may also have a gloss of greater than or equal to 5, in particular greater than or equal to 10, especially greater than or equal to 15, more particularly greater than or equal to 20, especially greater than or equal to 25, greater than or equal to 30 or even about 50.

The term “gloss” denotes the gloss as may be measured by the following method, using a gonioreflectometer machine, for instance the GRM-2000 machine (from Micromodule), using an azimuth illumination angle of 30° relative to the normal of the sample, a specular reflection (R) detection angle of −30° and a diffuse reflection (D) detection angle of 0°.

A support of rectangular foam type 40×70 mm in size is made using a brick-red foam (L*=37±3; a*=15±2; b*=11±2 in the CIE L*a*b*1976), made of Neoprene® 3 mm thick, which has an adhesive face, especially a foam known under the trade reference RE40×70 C/C 212B 1 skin, sold by the company Joint Technique Lyonnais Ind.

A transparent adhesive plaster sold by the company 3M under the trade reference Blenderm® FH 5000-55113, having a wear quality such that the application of a composition to this coating makes it possible to simulate application to the skin or mucous membranes, the sensation on application and the resulting color being similar even if the film gives poor coverage, is attached to the face opposite the adhesive face of this support.

The foam support bearing the transparent adhesive plaster is then attached, by bonding by means of its adhesive face, to a metal plate 40×70 mm in size. The assembly consisting of the support bonded to the metal plate forms a specimen.

The operator produces a total of 5 specimens identical to the one described above.

One embodiment of the process for evaluating the gloss will now be described.

The operator places the specimen on a hotplate set at a temperature of 38.5° C., for example a hotplate of the type N81076 sold by the company Fisher Bioblock, and waits for the face of the support bearing the adhesive coating to reach a temperature of 33±1° C.

Once the support is at the desired temperature, the operator manually applies a film about 15 μm thick of the cosmetic product to the Blenderm® coating.

The cosmetic product, which is, for example, a lipstick, was stored at 24±2° C.

The action performed by the operator to deposit the film of product consists of a to-and-fro motion, so as to obtain a uniform deposit. The application of the product to the support is preferably performed so as to be as representative as possible of the real conditions of application of the product. The same test product is applied in an identical manner to the five same specimens prepared previously.

The film of product is left to dry, the specimen being placed on the hotplate, such that the support remains at 33±1° C. for 10 minutes.

The intensity of the specular reflection and that of the diffuse reflection of the film of product are measured at the incidences specified previously, for each of the five specimens.

From the measured values, the gloss Glossᵢ is calculated for each specimen via the ratio R/D for this specimen. The weighting of the specular reflection measurement, generally used alone to characterize the gloss, by the diffuse reflection measurement (representative of the color/lightness of the sample) allows the visual perception of the gloss to be better appreciated.

The mean gloss value G̅l̅o̅s̅s̅ may finally be calculated:

$$\overline{Gloss} = \frac{1}{N} {\sum }_{i} {Gloss}_{i}$$

$$standard deviation \text{:} {σ}_{Gloss} = {\sqrt{\frac{N {\sum }_{i} {Gloss}_{i}^{2} - ( {\sum }_{i} {Gloss}_{i} )}{N ( N - 1 )}}}^{2}$$

$$95 \% confidence interval \text{:} Gloss \pm 1.96 \sqrt{\frac{{σ}_{Gloss}}{N}}$$

where N denotes the number of measurements, i.e. 5 in the present case.

Needless to say, the compositions according to the invention may be obtained according to the preparation processes conventionally used in cosmetics or dermatology.

The composition may be in the form of a paste or a cream. It may be an emulsion, especially an oil-in-water or water-in-oil emulsion, a solid or soft anhydrous gel, or else in the form of loose or compacted powder, and even in two-phase form. According to one particular variant, it is in the form of an emulsion.

It may also be in a solid form, compacted or cast as a stick or in a dish, or in pasty or liquid form.

Advantageously, it is in solid form, i.e. in hard form (which does not flow under its own weight), especially cast or compacted, for example as a stick or a dish.

According to one particular variant of the invention, it is in the form of lipsticks or lip balms.

A composition in accordance with the invention may also be in the form of a “liquid gloss”. The term “liquid gloss” and similarly the terms “liquid lipstick” and “lip gloss” denote a fluid product intended to be applied to the lips.

The compositions according to the invention may be in the form of a colored or uncolored product or in the form of an antisun product. They may especially contain cosmetic active agents. They may then be used as a care or treatment base for the lips, for instance lip balms, for protecting the lips against the cold and/or sunlight and/or the wind.

As cosmetic active agents that may be used in the invention, mention may be made of sunscreens, vitamins A, E, C and B3, provitamins, for instance D-panthenol, calmative active agents, for instance α-bisabolol, aloe vera or allantoin, plant extracts or essential oils, protective or restructuring agents, for instance ceramides, refreshing active agents, for instance menthol and derivatives thereof, emollients (cocoa butter), moisturizers (arginine PCA), anti-wrinkle active agents and essential fatty acids, and mixtures thereof.

The composition of the invention may also be in the form of a lip makeup product, for instance a lipstick or a lip gloss, optionally having care or treating properties.

The examples below are given by way of illustration without being limiting in nature.

### EXAMPLES

In the examples, the compounds can be independently one from the other be chosen from compounds approved for food use. According to one embodiment they can independently advantageously be food grade compounds as defined here-above.

The following compounds can be used in the examples:
the caprylic/capric acid triglycerides sold by Stepan under the trademark NEOBEE M-5,
the sucrose acetate isobutyrate sold by Eastman under the trademark Eastman SAIB-100,
the deodorized apricot kernel oil sold by Nestlé under the trademark Apricot Kernel Oil Type C,
the microcrystalline wax sold by Paramelt under the trademark Micro Wax HW,
ozokerite, sold by Strahl & Pitsch, under the trademark Ozokerite Wax SP 1020,
beeswax, candelilla wax and carnauba wax sold by Strahl & Pitsch under the trademarks Cerabeil Lor, Candelilla Wax SP 75 and Cerauba T1,
the hydrogenated plant oils sold by Karlshamns under the trademark AKOGEL,
the rice starch sold by REMY under the trademark Remy RDI,
the canola oil sold by Karlshamn under the trademark AKOREX L, and
the isopropyl myristate sold by Stearinerie Dubois under the trademark Myristate d′isopropyle (DUB IPM).

The sticks of lipstick illustrated by the examples below are prepared according to the following protocol:

The waxes, the pasty compounds and the oils are melted at 100° C. The ground pigmentary material containing the iron oxides and/or the aluminum lakes is incorporated therein and the mixture is then stirred for 45 minutes. At the end of the stirring period, the pearls and optionally the flavoring are added. The mixture is poured into a lipstick mold preheated to 42° C. The mold is then placed in a refrigerator until the temperature of the mold reaches 2° C. The sticks are then removed from the molds and the products are stored at 20° C. for 24 hours.

The properties in terms of gloss, wear, and covering power of the compositions are good.

### Example 1

Lipstick

Its composition is as follows:

| | weight % |
|---|---|
| Microcrystalline wax | 11.25 |
| Carnauba wax | 3.75 |
| Caprylic/capric acid triglycerides | 76 |
| Brown iron oxide | 8 |
| Yellow iron oxide | 0.3 |
| Titanium oxide | 0.7 |
| Total | 100 |

### Example 2

Lipstick

Its composition is as follows:

| | weight % |
|---|---|
| Microcrystalline wax | 11.25 |
| Carnauba wax | 3.75 |
| Caprylic/capric acid triglycerides | 66 |
| Brown iron oxide | 8 |
| Yellow iron oxide | 0.3 |
| Titanium oxide | 0.7 |
| Sucrose acetate isobutyrate | 10 |
| Total | 100 |

### Example 3

Lipstick

Its composition is as follows:

| | weight % |
|---|---|
| Ozokerite wax | 9.75 |
| Beeswax | 3.25 |
| Caprylic/capric acid triglycerides | 32 |
| Brown iron oxide | 8 |
| Yellow iron oxide | 0.3 |
| Titanium oxide | 0.7 |
| Sucrose acetate isobutyrate | 35 |
| Hydrogenated plant oil | 10 |
| Rice starch | 1 |
| Total | 100 |

### Example 4

Lipstick

Its composition is as follows:

| | weight % |
|---|---|
| Ozokerite wax | 9 |
| Beeswax | 3 |
| Caprylic/capric acid triglycerides | 27 |
| Brown iron oxide | 8 |
| Yellow iron oxide | 0.3 |
| Titanium oxide | 0.7 |
| Sucrose acetate isobutyrate | 35 |
| Hydrogenated plant oil | 16 |
| Rice starch | 1 |
| Total | 100 |

### Example 5

Lipstick

Its composition is as follows:

| | weight % |
|---|---|
| Ozokerite wax | 9 |
| Beeswax | 3 |
| Caprylic/capric acid triglycerides | 6.75 |
| Brown iron oxide | 8 |
| Yellow iron oxide | 0.3 |
| Titanium oxide | 0.7 |
| Sucrose acetate isobutyrate | 35 |
| Apricot oil | 20.25 |
| Hydrogenated plant oil | 16 |
| Rice starch | 1 |
| Total | 100 |

### Example 6

Lipstick

| | weight % |
|---|---|
| canola oil | 19.32 |
| Sucrose acetate isobutyrate | 37.5 |
| Ditertiobutyl 4-hydroxytoluene (BHT) | 0.1 |
| Iron oxides | 1.36 |
| Alumina | 2.16 |
| Titanium dioxide | 1.82 |
| Yellow 6 lake | 0.86 |
| Red 27 lake | 0.29 |
| Red 7 | 1.51 |
| Microcrystalline wax | 1 |
| Caprylic/capric acid triglycerides | 6.58 |
| Hydrogenated vegetable oil | 16 |
| Candelilla wax | 4 |
| Carnauba wax | 6.5 |
| Rice starch | 1 |
| Total | 100 |

### Examples 7 to 13

Table 1 below presents seven lipstick formulations incorporating, as dyestuffs, lakes and pearls approved for food use.

The lipstick compositions contain a white substance used in combination with the various mixtures of pearls and lakes. The white substance composition is as follows:

| | weight % |
|---|---|
| Ozokerite | 9.9 |
| Beeswax | 3.4 |
| Caprylic/capric acid triglycerides | 7.4 |
| Apricot kernel oil | 22.3 |
| Sucrose acetate isobutyrate | 38.4 |
| Hydrogenated plant oil | 17.6 |
| Rice starch | 1 |
| Total | 100.0 |

| Starting | Example 7 | Example 8 | Example 9 | Example 10 | Example 11 | Example 12 | Example 13 |
|---|---|---|---|---|---|---|---|
| material | weight % | weight % | weight % | weight % | weight % | weight % | weight % |
| white | 91 | 85.81 | 87.06 | 79.61 | 87.71 | 86.92 | 79.29 |
| substance | | | | | | | |
| brown iron | | | | 3.38 | | 0.36 | |
| oxide | | | | | | | |
| yellow iron | | | | | | | 2.7 |
| oxide | | | | | | | |
| titanium | 1.06 | 0.77 | 0.21 | 3.38 | 1.12 | 0.72 | 5.4 |
| oxide | | | | | | | |
| brown, | | | | | 0.75 | | 1.8 |
| yellow iron | | | | | | | |
| oxide | | | | | | | |
| Candurin red | | 7.26 | | | | | |
| amber pearl | | | | | | | |
| Candurin | | | | | 4.17 | | |
| silver sparkle | | | | | | | |
| pearl | | | | | | | |
| Candurin | | | | | 2.08 | | |
| silver sheen | | | | | | | |
| pearl | | | | | | | |
| Candurin | | | | | 4.17 | | |
| gold shimmer | | | | | | | |
| pearl | | | | | | | |
| Candurin red | | | 9.52 | | | | |
| lustre pearl | | | | | | | |
| Candurin | | | | | | | 2.16 |
| brown amber | | | | | | | |
| pearl | | | | | | | |
| Candurin red | | | | | | 12 | |
| shimmer | | | | | | | |
| pearl | | | | | | | |
| Candurin | | | | 12.5 | | | 8.65 |
| silver lustre | | | | | | | |
| pearl | | | | | | | |
| FD & C Blue | 0.53 | 1.28 | 0.21 | | | | |
| 1 aluminum | | | | | | | |
| lake | | | | | | | |
| FD & C | | 4.37 | | 1.13 | | | |
| Yellow 6 | | | | | | | |
| aluminum | | | | | | | |
| lake | | | | | | | |
| FD & C Red | 7.41 | 0.51 | 3 | | | | |
| 40 aluminum | | | | | | | |
| lake | | | | | | | |
| total: | 100 | 100 | 100 | 100 | 100 | 100 | 100 |

The candurin pigments are sold by MERCK.

### Example 14

Compact foundation formulation:

| | weight % |
|---|---|
| Beeswax | 2 |
| Carnauba wax | 4 |
| Isopropyl myristate | 53 |
| Talc | 31 |
| Iron oxide | 3 |
| Titanium dioxide | 7 |

### Example 15

Cast foundation formulation:

| | weight % |
|---|---|
| Beeswax | 4 |
| Carnauba wax | 15 |
| Isopropyl myristate | 35 |
| Caprylic/capric acid triglycerides | 12 |
| Talc | 26 |
| Iron oxide | 3 |
| Titanium dioxide | 5 |

1-20. (canceled)

21. A cosmetic process for making up and/or caring for keratin material(s), comprising the application, on contact with a keratin material, of a cosmetic composition comprising at least 75% by weight of compound(s) approved for food use relative to its total weight, and having a covering power of greater than or equal to 30.

22. The process as claimed in claim 21, in which the composition comprises at least 80%, by weight of compound(s) approved for food use relative to its total weight.

23. The process as claimed in claim 21, in which the composition has a covering power at least greater than or equal to about 50.

24. The process as claimed in claim 21, in which the composition comprises at least one fatty phase.

25. The process as claimed in claim 24, in which the fatty phase comprises at least one compound selected from the group consisting of oils, waxes and pasty fatty substances, and mixtures thereof.

26. The process as claimed in claim 25, in which the composition comprises from 0.1% to 99% by weight relative to its total weight, of oil(s) referenced in the Codex alimentarius.

27. The process as claimed in claim 25, in which the oil is selected from the group consisting of oils comprising at least one fatty acid selected from the group consisting caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid and nervonic acid, and a mixture thereof.

28. The process as claimed in claim 25, in which the oil is selected from the group consisting of heptanoic or octanoic triglycerides, groundnut oil, babassu oil, coconut oil, grapeseed oil, cottonseed oil, corn oil, corn germ oil, mustard seed oil, palm oil, rapeseed oil, sesame seed oil, soybean oil, sunflower oil, wheatgerm oil, apricot oil, mango oil, castor oil, shea oil, avocado oil, olive oil, sweet almond oil, peach kernel oil, hazelnut oil, walnut oil, macadamia oil, jojoba oil, alfalfa oil, poppy seed oil, pumpkin oil, marrow oil, blackcurrant oil, evening primrose oil, millet oil, barley oil, quinoa oil, canola oil, rye oil, safflower oil, candlenut oil, passionflower oil, musk rose oil or shea butter oil or else caprylic/capric acid triglycerides, and mixtures thereof.

29. The process as claimed in claim 25, in which the composition comprises from 1% to 50% by weight of wax(es) approved for food use.

30. The process as claimed in claim 25, in which the wax is selected from the group consisting of carnauba wax, candelilla wax, beeswax, microcrystalline waxes, ozokerite and rice wax, and mixtures thereof.

31. The process as claimed in claim 25, in which the composition comprises at least one pasty compound selected from the group consisting of polyol esters.

32. The process as claimed in claim 21, in which said composition comprises at least one aqueous phase.

33. The process as claimed in claim 32, in which the composition comprises from 0.1% to 25% by weight of an aqueous phase, relative to its total weight.

34. The process as claimed in claim 21, in which the composition is in the form of an emulsion or a stick.

35. The process as claimed in claim 21, in which the composition also comprises at least one dyestuff.

36. The process as claimed in claim 35, in which said dyestuff is selected from the group consisting of the compounds referenced in the Codex alimentarius and pearls consisting of materials referenced in the Codex alimentarius.

37. The process as claimed in claim 21, in which the composition also has a gloss value at least greater than or equal to about 5.

38. The process as claimed in claim 21, in which the composition is applied to the skin and/or the lips.

39. The process as claimed in claim 21, in which the composition is a makeup and/or care product for the skin and/or the lips.

40. A cosmetic composition for making up and/or caring for keratin material(s) as defined according to claim 21.
